(19) United States
(12) Patent Application Publication
(10) Pub. No.: US 2017/0333661 A1
(43) Pub. Date: Nov. 23, 2017

(54) RESPIRATORY THERAPY APPARATUS

(71) Applicant: SMITHS MEDICAL INTERNATIONAL LIMITED, Kent (GB)

(72) Inventors: Paul James Bennett, Bedfordshire (GB); Anthony Lucio Belisario, Luton (GB); Robert James Burchell, Hertfordshire (GB); Mohammad Qassim Mohammad Khasawneh, Milton Keynes (GB); Mark Charles Oliver, Hertfordshire (GB)

(73) Assignee: SMITHS MEDICAL INTERNATIONAL LIMITED, Kent (GB)

(21) Appl. No.: 15/533,134

(22) PCT Filed: Nov. 10, 2015
(86) PCT No.: PCT/GB2015/000298

§ 371 Date: Jun. 5, 2017

(30) Foreign Application Priority Data
Dec. 11, 2014 (GB) 1422001.6

(51) Int. Cl. A61M 16/08; A61M 16/00; A63B 23/18

(52) U.S. Cl. CPC A61M 16/0866; A63B 23/18; A61M 16/0006; A61M 2205/583

(57) ABSTRACT

An expiratory, vibratory therapy device (100, 200 300, 400) includes a compliance meter (104, 204, 304, 404) having a cylinder (110, 210, 310, 410) with a piston (111, 211, 411) movable along its length. The piston is urged by a spring (116, 226) to one end of the cylinder. The opposite end of the cylinder has an air inlet (120, 123, 219) normally closed by a springloaded occluder (129). The occluder (129) is moved to open the air inlet when the patient exhales in the prescribed manner. This allows some air to enter the cylinder and enables the piston to be displaced. The piston is coupled to a flag (106, 206, 306, 406) visible to the patient. When the correct number of prescribed breaths have been made the piston displaces the flag to indicate completion of the therapy.

This invention relates to respiratory therapy devices of the kind arranged to produce a resistance to respiratory flow through the device.

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis and atelectasis. More recently, such devices that provide an alternating resistance to flow have been found to be particularly effective. One example of such a device is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. U.S. Pat. No. 8,534,284 describes a device with an interrupter valve driven by pressurised gas delivered to the apparatus. The speed of the valve is dependent on the back pressure created by expired breaths from the patient. Other vibratory respiratory therapy devices are available, such as “Quake” manufactured by Thayer, “AeroPEP” manufactured by Monaghan, “TheraPEP” manufactured by Smiths Medical and “IPV Percussionator” manufactured by Percussionaire Corp. These devices generate vibratory positive pressures mechanically and fluctuating exhalation flows that help overcome the inertia and stiction of the sputum within the bronchi and lower passages of the lung. This enhances mucociliary clearance. Alternative apparatus such as “CoughAssist” manufactured by Philips is also available. Respiratory therapy devices can instead provide an alternating resistance to flow during inhalation.

Although respiratory therapy devices can be highly effective at treating respiratory impairments, the relief obtained is dependent, on how closely the patient adheres to the prescribed treatment regime: how regularly he uses the device and the manner in which the device is used. Patients are trained to use the devices by a clinician in a hospital but it is essential that the devices are used regularly by the patient in the prescribed manner at home where there is no clinical supervision. The problem, however, is that the patient may not use the device as prescribed when unsupervised, outside a clinical environment. The clinician is unable to determine whether any lack of improvement in the patient's condition is due to his failure to adhere to the treatment regime or other factors so this makes control of the patient's condition very difficult.

It is an object of the present invention to provide an alternative respiratory therapy device.

According to one aspect of the present invention there is provided a respiratory therapy device of the above-specified kind, characterised in that the device includes an indicator arranged to provide an indication of the extent of correct use of the device, that the indicator includes an air chamber with an air inlet and a displaceable wall, an arrangement for urging the displaceable wall relative to the chamber to increase the volume of the chamber, that the device includes a mechanism coupled with the air inlet and acted on by patient breathing through the device, that the mechanism is arranged to open the air inlet when the patient breathes through the device in a prescribed manner, thereby allowing air to enter the chamber and the wall to be displaced, and that the movement of the wall is coupled with an indicator flag such that use of the device causes the wall to be displaced and the flag to change.

The therapy device is preferably arranged to produce an alternating resistance to respiratory flow and may be arranged to produce an alternating resistance to expiratory flow through the device. The displaceable wall may be provided by a piston movable along a cylinder. The arrangement for urging the displaceable wall is preferably resilient means, such as a helical spring. The device preferably includes a one-way valve arranged to allow air to flow out of the air chamber when the device is set before use. The mechanism coupled with the air inlet may include a spring-loaded valve element. The spring force acting on the valve element is preferably adjustable. The device preferably includes an adjustable flow regulator arranged to regulate flow of air into the air inlet. The indicator flag may be displaced linearly or rotatably. The indicator flag may be arranged to be displaced continuously or only when the displaceable wall is close to its limit of displacement towards the end of a breath.

A vibratory PEP device according to the present invention will now be described, by way of example, with reference to the accompanying drawings, in which:

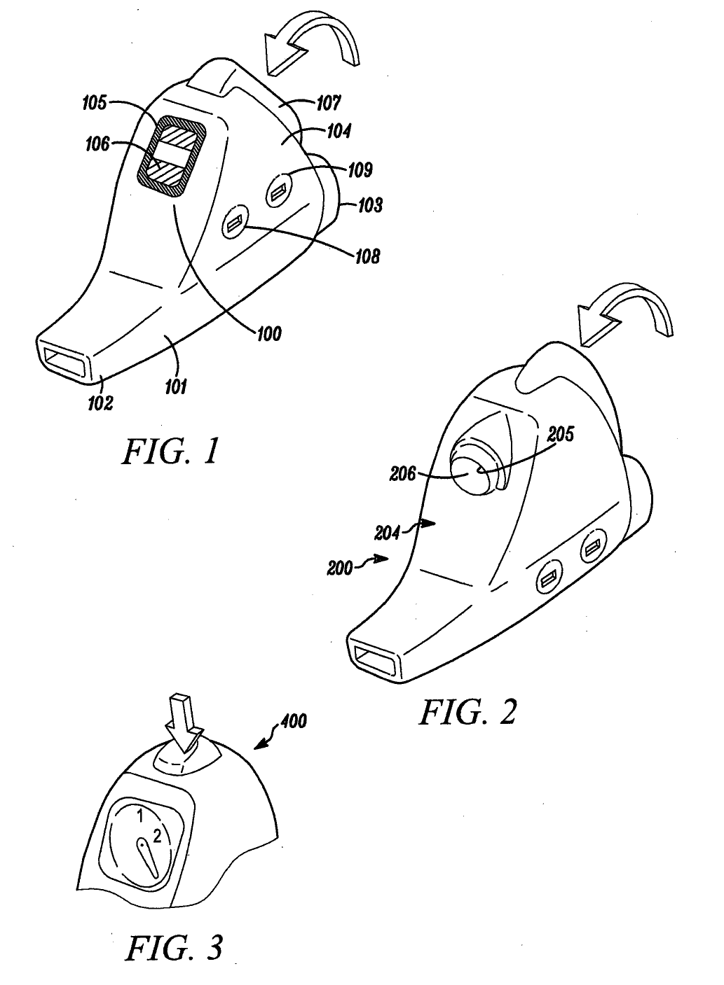

FIG. 1 is a perspective view of the outside of a first embodiment of the device;

FIG. 2 is a perspective view of the outside of a second embodiment of the device;

FIG. 3 is a perspective view of the outside of a part of a third embodiment of the device;

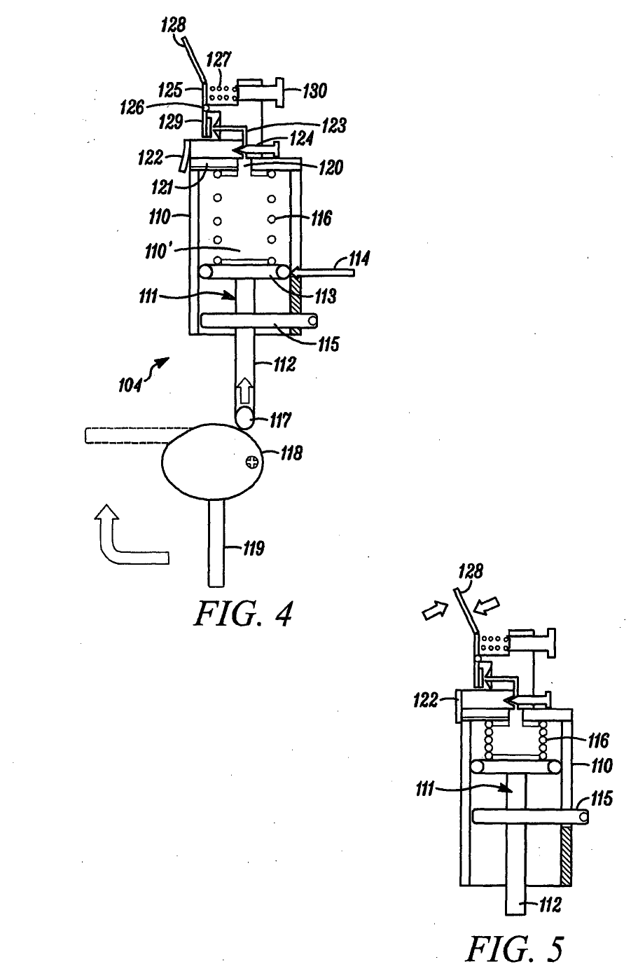

FIGS. 4 to 7 show schematically the construction and operation of the first embodiment;

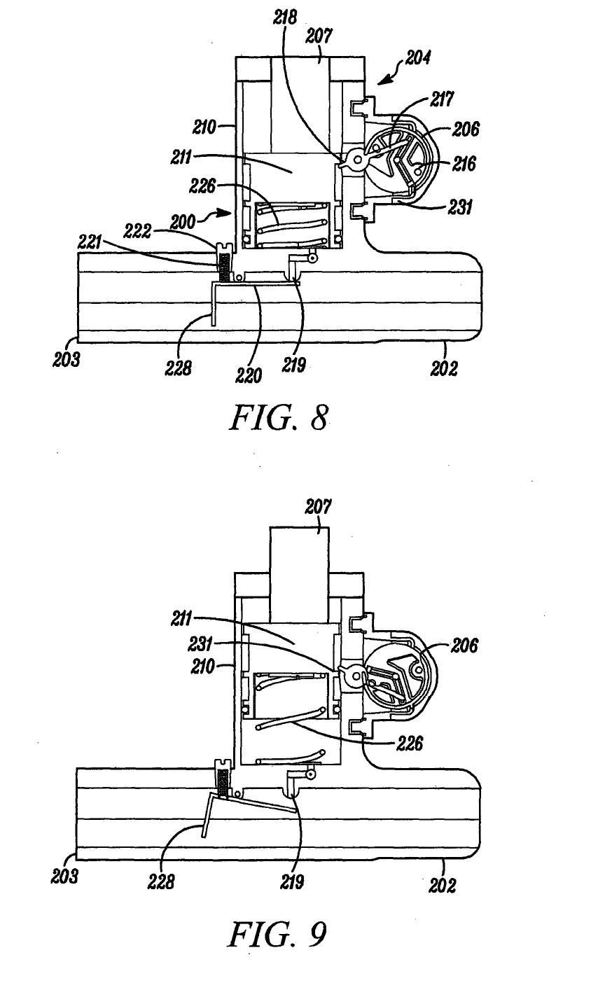

FIGS. 8 and 9 show schematically the construction and operation of the second embodiment;

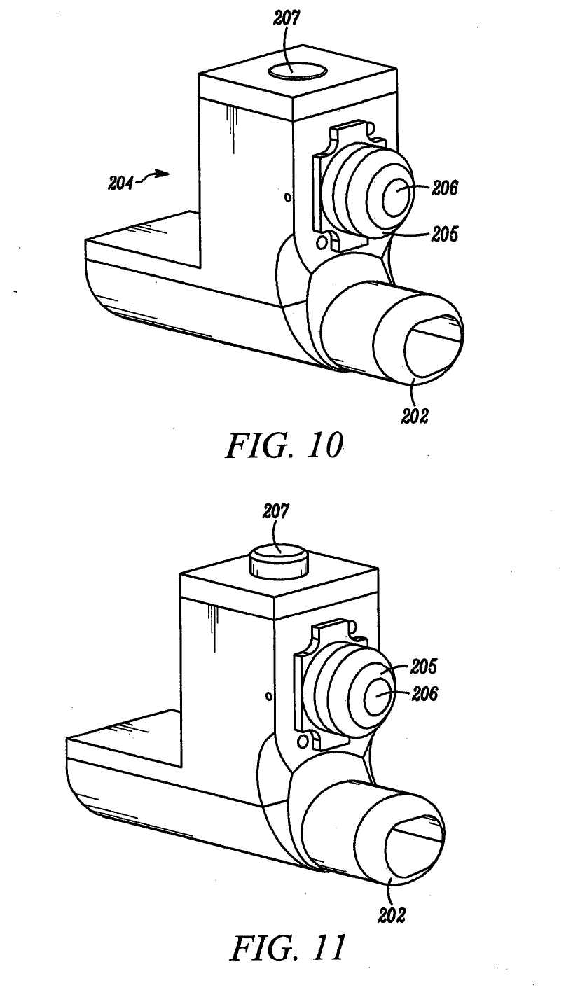

FIGS. 10 to 12 are perspective views of the outside of the second embodiment at different conditions;

FIG. 13 is a perspective view of the rear, outlet end of the second embodiment;

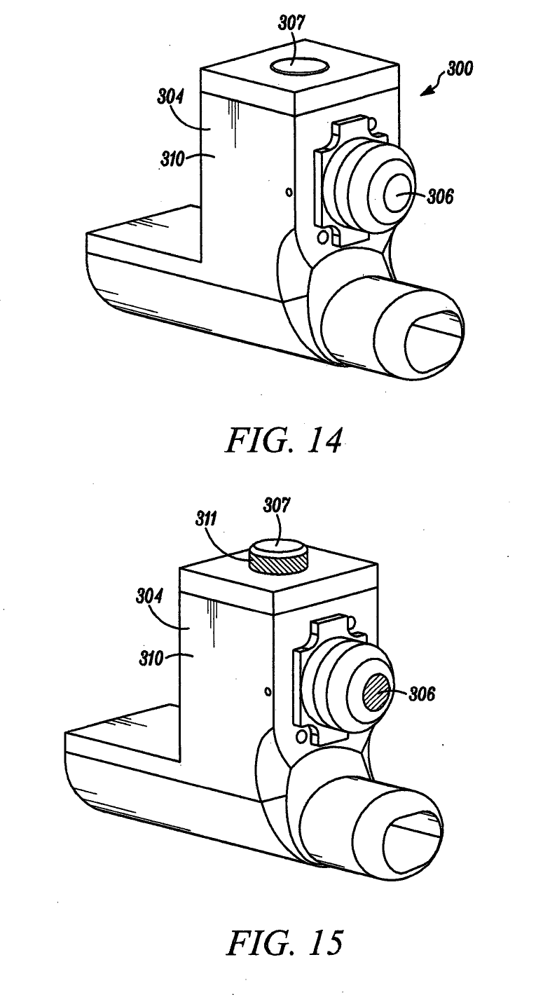

FIGS. 14 to 17 are perspective views of the outside of the third embodiment at different conditions;

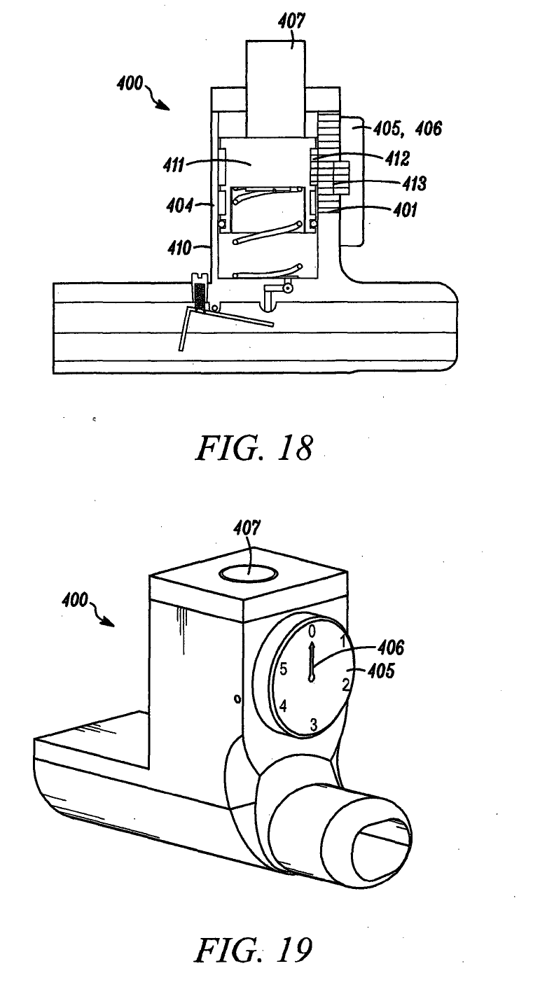

FIG. 18 shows schematically the construction of the fourth embodiment of the invention; and

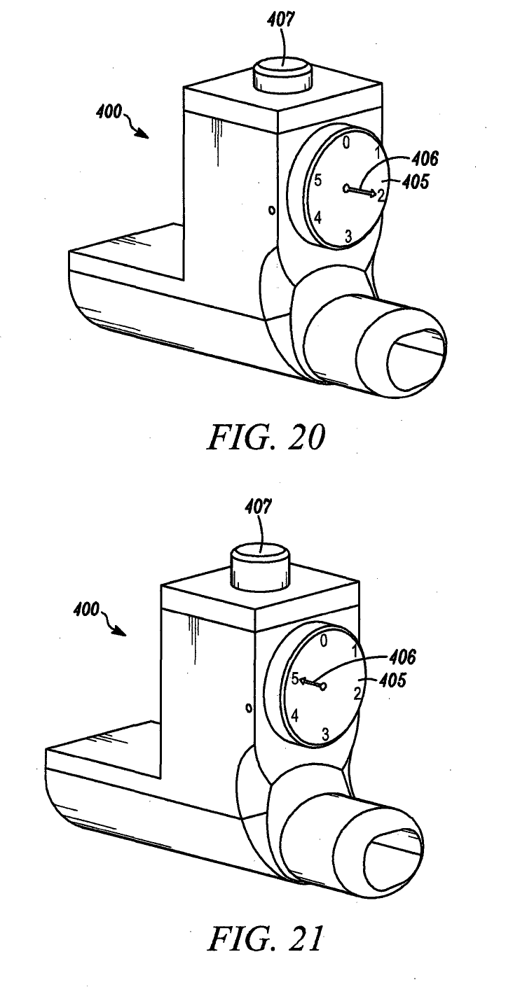

FIGS. 19 to 21 are perspective views of the outside of the fourth embodiment at different conditions.

With reference first to FIG. 1 the respiratory therapy device 100 is of a kind that produces vibration within the user's lungs similar to an Acapella respiratory expiratory therapy device as sold by Smiths Medical. The device 100 has a housing 101 with a patient inlet or mouthpiece 102 at its forward end and an outlet opening 103 at its rear end. Between the inlet 101 and outlet 102 the device 100 has a mechanism (not shown) that provides an alternating, vibratory or oscillating resistance to expiratory flow. The mechanism may be of the kind described in U.S. Pat. No. 6,581,598 but various alternative therapy mechanisms could be used, which need not involve an alternating resistance. An understanding of the therapy mechanism is not essential to an understanding of the present invention. The upper part of the housing 101 contains a pneumatic compliance meter or indicator identified generally by the numeral 104. The indicator 104 has a window 105 facing the forward end of the device 100 so that it can be seen by the user when using the device. Within the window 105 there is a flag or indicator 106 that is moved by a compliance mechanism to indicate to the user that he has completed a breath correctly. The compliance meter 104 also includes a reset button 107 that the user pushes in to reset its operation prior to the start of each breath. The meter 104 further includes two adjustment controls, namely a flow trigger level adjustment 108 and a time adjustment 109.

Operation and further details of the construction of the compliance meter 104 will now be described with reference additionally to FIGS. 4 to 7, which illustrate its mechanism from an initial setting before use through three subsequent states at successive times during a single breath.

Referring first to FIG. 4, the mechanism includes a cylinder 110 having a piston assembly 111 with a piston rod 112 projecting downwardly out of the cylinder and connected at its upper end with a piston head 113. The piston head 113 has a piston ring or rolling diaphragm 114 providing a displaceable wall that makes a sealing, sliding or rolling contact with the inside of the cylinder 110. The cylinder 110 and piston head 113 define between them an air chamber 110′ of variable size. The piston rod 112 supports a laterally projecting arm 115 that is coupled with the flag 106 so that movement of the piston 111 effects linear displacement of the flag along its length. The cylinder 110 also contains resilient means in the form of a helical compression spring 116 positioned between the upper face of the piston head 113 and the upper inside end of the cylinder and arranged to apply a force to the piston 111 urging it outwardly of the cylinder. Other arrangements could be used to apply a restoring force to the piston, such as including a mass. The spring 116 could be a parallel compression spring or a variable pitch conical spring in order to maintain a constant force at different extents of compression.

The lower end 117 of the piston rod 112 engages a rotatable cam 118 having an outwardly projecting lever 119 by which the cam can be rotated. The lever 119 is coupled with the reset button 107 so that pressing the reset button inwards rotates the lever and cam 118 clockwise and displaces the piston 111 upwardly from the outermost or lowest position shown in FIG. 4 to the innermost, highest or set position shown in FIG. 5.

The upper end of the cylinder 110 has a central gas opening 120 through which air can flow into and out of the cylinder. This central opening 120 communicates with a side venting passage 121 at the outer end of which is mounted a non-return flap valve 122, which provides a path for gas to flow out of the cylinder 110 but closes to prevent any gas flowing into the cylinder via the side passage. The opening 120 also communicates with an inlet passage 123, which includes an adjustable flow regulator or restrictor 124 coupled with the time adjustment control 109. The outer end of the inlet passage 123 is terminated by a spring-loaded valve element or occluder 129, which normally blocks the passage and prevents any air flowing via the passage into the cylinder 110. The occluder 129 is provided by the lower end of an arm 125 that is hinged about a horizontal axis at a point 126 along its length. A spring 127 bears on the upper end of the arm 125 urging it in an anticlockwise direction so that its lower end is urged to the right, against the opening of the inlet passage 123. The upper end of the arm 125 is angled and shaped to form a paddle 128 that is either exposed directly to gas flow along the housing 101, between the inlet 102 and outlet 103, or indirectly such as via a diaphragm and coupling. A screw-threaded adjustment boss 130 supports the right-hand end of the spring 127 so that the force required to lift the occluder off the inlet of the passage 123 can be adjusted and set appropriately. The adjustment boss 130 either provides the flow trigger adjustment 108 directly or is coupled with this. The arrangement of the occluder 129 is such that, if the expiratory flow of air through the device 100 or the pressure caused by the user is high enough, the force on the left-hand side of the paddle 128 will be sufficient to rotate the arm 125 clockwise and open the inlet passage 123 to allow air to flow into the cylinder 110.

The condition represented in FIG. 4 shows the compliance meter 104 at its rest position and the user just starting to reset the meter by actuating the button 107, before a breath, to start raising the piston 111 in the cylinder 110 against the action of the spring 116. The increased air pressure created in the cylinder 110 is sufficient to open the non-return valve 122 to allow air to vent from the cylinder but is insufficient to open the inlet occluder 129.

FIG. 5 shows the state of the compliance meter 104 after the reset button 107 has been fully depressed and released and before the user starts a breath. The spring 116 applies a force to the piston 111 tending to urge it outwardly but, because the non-return valve 122 is drawn inwardly to a sealing position by the reduced pressure within the cylinder 110, no air can flow into the cylinder and so the piston cannot move.

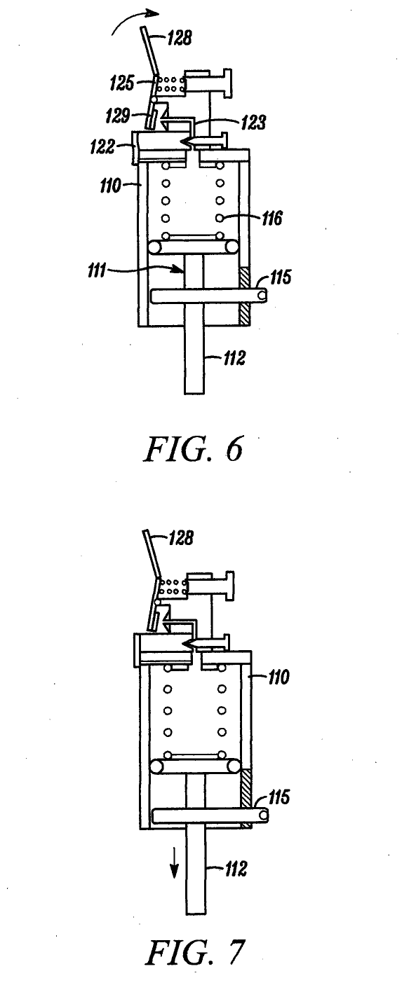

FIG. 6 shows the condition when the user starts a breath by exhaling through the device 100 in the prescribed manner to generate sufficient flow and pressure in the device for effective treatment. This flow or pressure applies a force to the left-hand side of the paddle 128 sufficient to rotate the arm 125 through a small angle clockwise enough to lift its lower end clear of the inlet of the passage 123. This allows air to flow along the passage 123 into the cylinder 110, which allows the piston 111 to be moved down by the spring 116. The flow or pressure required to open the occluder 129 is controlled by suitable adjustment of the flow trigger adjustment 108 via the boss 130. The rate of flow of air into the cylinder 110 when the inlet passage 123 is open is controlled by the time adjustment control 109 via the flow restrictor 124. The vibratory nature of the therapy device 100 means that an alternating flow and pressure is built up in the device so the pressure applied to the paddle 128 is of an alternating fashion causing the occluder 129 to open and close at the same frequency. This allows air to flow into the cylinder 110 and allows the piston 111 to move outwardly in a stepped manner until it reaches its fully extended position at the end of one expiratory breath as shown in FIG. 7. As the piston 111 moves outwardly it displaces the arm 115 and the indicator flag 106 to which it is coupled. The flag 106 may be marked in various ways such as with regions of different colour so that, for example, at the start of a breath when the meter is reset, a red colour portion of the flag is visible in the window 105. As the breath continues, the flag 106 is moved progressively and gradually through a position where an orange portion is visible to a position where a green portion is visible when the breath has been completed at the prescribed flow and pressure levels.

It will be appreciated that this arrangement is such that if the user does not use the device correctly, so that insufficient flow or pressure is created, the occluder 129 will not be opened and the indicator flag 106 will not be advanced.

An alternative embodiment will now be described with reference to FIGS. 2 and 8 to 13. This embodiment could have a similar vibratory therapy mechanism to that described with reference to FIGS. 1 and 4 to 7 but differs in that the compliance indicator or meter 204 has an indicator flag 206 of spherical or drum form. The flag 206 is coupled with a linear to rotating mechanism 216 including a hinged arm 217 attached with the mechanism at one end and having its other free end 218 projecting into the cylinder 210. The piston 211 has a surface formation in the form of a projection or ledge 231 that is located towards its lower end to engage the free end 218 of the arm 217 only when the piston is at, or close to, its fully extended upper or outer position. The cylinder 210 opens at its lower end into the air passage through the device 200 via an inlet 219. The inlet 219 is normally occluded by the right-hand end of an arm 220, which is hinged between its ends and is urged down towards its left-hand end by an adjustable spring 221. The arm 220 has a downwardly-depending paddle 228 projecting laterally into flow stream through the device 200. The paddle 228 could include a non-return valve to allow inspiratory flow.

FIGS. 8 and 10 show the state of the meter 204 after having been set but before use. The piston 211 is fully down with its spring 212 fully compressed and with its reset button 207 flush with the top of the cylinder 210. The arm 220 bears on the inlet 219 so that it is sealed closed and no air can flow into the cylinder 210, thereby preventing the piston 211 rising in the cylinder. In this state the spherical indicator flag or ball 206 shows a neutral colour or marking in the window 205 to indicate that the breath has not yet been fully completed.

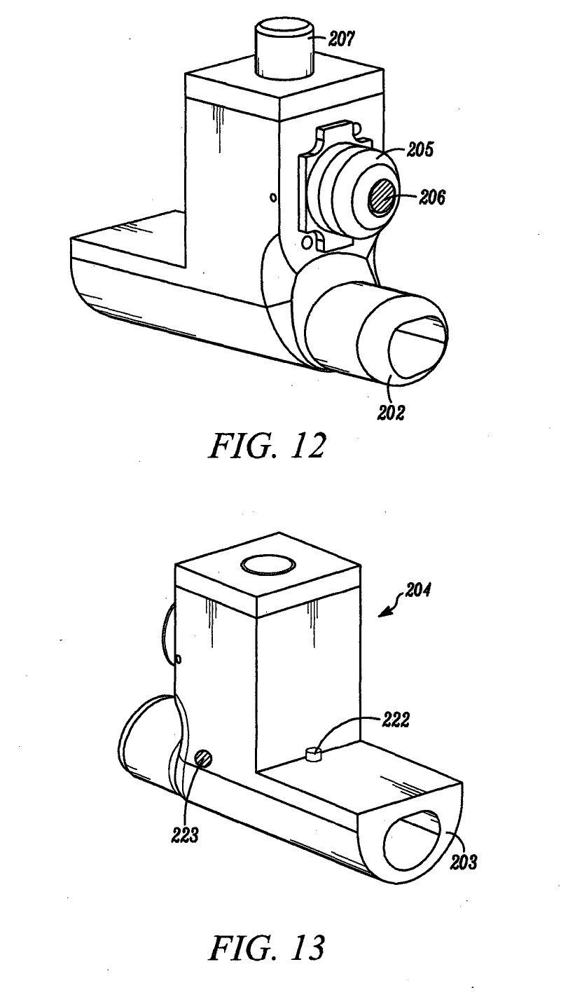

As the user starts a breath by exhaling through the device 200, if the exhalation force is sufficiently forceful, it will apply sufficient pressure to the paddle 228 to cause the arm 220 to rotate clockwise. It can be seen that this will cause the right-hand end of the arm 220 to move down away from the inlet 219, thereby allowing air to flow through the inlet into the cylinder 210. This allows the piston 211 and reset button 207 to be moved up the cylinder 210 by the spring 226 a small distance, as shown in FIG. 11. During this initial upward movement there is no change in the appearance of the flag 206 since the piston 211 is free to slide past the free end 218 of the arm 217 without displacing the arm. FIG. 11 illustrates how the reset button 207 is elevated proud of the upper part of the cylinder 210 a small distance. It is only when the piston 211 is close to its maximum upward extension as shown in FIGS. 9 and 12 that the ledge 231 on the piston 211 contacts the underside of the free end 218 of the arm 217 and displaces this up, thereby displacing the opposite end of the arm down. This rotates the flag ball 206 clockwise to bring a differently marked or coloured region of the flag into view through the window 205. In this arrangement the flag 206 moves more rapidly between two different states when the breath has been completed as prescribed.

FIG. 13 shows two adjustable settings controls similar to those on the device shown in FIG. 1. The centrally positioned control 222 is a screw coupled to the spring 221 that bears on the upper side of the flow paddle arm 220. This can be altered to adjust the flow threshold at which the arm 220 is displaced to allow air into the cylinder 210. The other control 223 shown on one side of the device 200 is a screw control for a flow restrictor located in the path between the inlet 219 and the cylinder 210 (not visible in the drawings). By adjusting this control 223 the rate of flow of air into the cylinder 210, when the inlet 219 is open, can be altered, thereby altering the speed at which the piston 211 moves up the cylinder and hence the time taken for the indicator to change appearance.

It is not essential that the arrangement shown in FIG. 2 has an indicator with just two different states. Instead, the same mechanism could be arranged such that continued upward displacement of the piston causes further rotation of the flag to bring different colour regions into view through the window as each breath progresses.

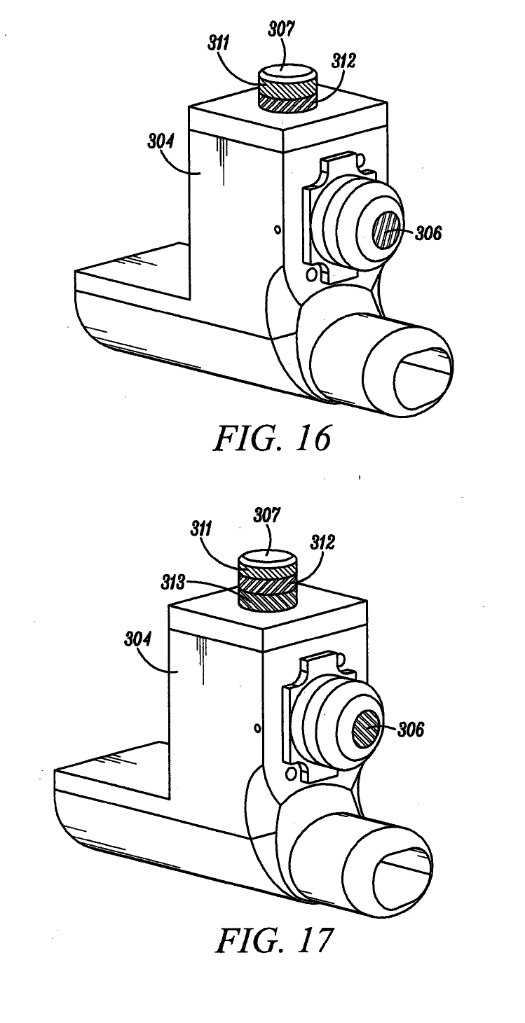

FIGS. 14 to 17 illustrate a device 300 with an indicator 304 where the indicator flag 306 has four different coloured or otherwise differently marked regions. These regions are visible one after the other as the breath progresses. FIG. 14 illustrates an initial starting state with a white colour region of the flag 306 visible. FIG. 15 illustrates a subsequent state with a blue colour region on the flag 306 being visible instead. FIG. 16 illustrates the next state where the flag 306 changes appearance to a green colour. FIG. 17 illustrates the final state at completion of the breath where the flag 306 appears as an orange colour. In this device the reset button 307 is marked around its cylindrical surface with three annular bands of colour or other markings 311, 312 and 313 to match those on the flag 306 so that when the flag changes appearance from white to blue the reset button projects from the cylinder 310 by a distance sufficient to reveal only the uppermost band 311 of a blue colour. Continued exhalation raises the reset button 307 further to reveal successive bands of green 312 and orange 313, with all three coloured bands being visible when the therapy breath has been completed.

With reference now to FIGS. 3 and 18 to 21 there is shown a device 400 with an indicator mechanism 404 similar to that of the arrangement of FIGS. 8 to 13 except that instead of a spherical, ball flag the indicator takes the form of a fixed dial 405 and a rotating pointer 406. The dial 405 is circular and marked around its edge with a scale of numerals. The pointer 406 is mounted at one end centrally of the dial and is rotated so that its outer end points to positions around the scale depending on the duration of correct exhalation. In its initial position (FIG. 19) immediately after setting the device 400 by fully depressing the reset button 407 the pointer 406 points vertically to the “0”. As the device 400 is used, the reset button 407 rises and the pointer 406 rotates clockwise through the position shown in FIG. 20 where the pointer points to the “2” to the final position shown in FIG. 21 where the pointer points to the “5”. FIG. 18 shows the mechanism 401 by which linear movement of the piston 411 is converted to rotation of the pointer 406. The piston 411 carries a toothed rack 412 extending along its length, which is engaged by a pinion and gear train 413 coupled to the spindle of the pointer 406, so that the pointer is rotated clockwise as the piston moves up along the cylinder.

The invention is not confined to vibratory or expiratory therapy devices but could be used with inspiratory therapy devices and those without any vibratory effect.

1-13. (canceled)

14. A respiratory therapy device arranged to produce a resistance to respiratory flow through the device, characterised in that the device includes an indicator to provide an indication of the extent of correct use of the device, that the indicator includes an air chamber with an air inlet and a displaceable wall, an arrangement for urging the displaceable wall relative to the chamber to increase the volume of the chamber, that the device includes a mechanism coupled with the air inlet and acted on by patient breathing through the device, that the mechanism is arranged to open the air inlet when the patient breathes through the device in a prescribed manner, thereby allowing air to enter the chamber and the wall to be displaced, and that the movement of the wall is coupled with an indicator flag such that use of the device causes the wall to be displaced and the flag to change.

15. A respiratory therapy device according to claim 14, characterised in that the device is arranged to produce an alternating resistance to respiratory flow.

16. A respiratory therapy device according to claim 15, characterised in that the device is arranged to produce an alternating resistance to expiratory flow through the device.

17. A respiratory therapy device according to claim 14, characterised in that the displaceable wall is provided by a piston movable along a cylinder.

18. A respiratory therapy device according to claim 14, characterised in that the arrangement for urging the displaceable wall is a resilient device.

19. A respiratory therapy device according to claim 18, characterised in that the resilient device is a helical spring.

20. A respiratory therapy device according to claim 14, characterised in that the device includes a one-way valve arranged to allow air to flow out of the air chamber when the device is set before use.

21. A respiratory therapy device according to claim 14, characterised in that the mechanism coupled with the air inlet includes a spring-loaded valve element.

22. A respiratory therapy device according to claim 21, characterised in that the spring force acting on the valve element is adjustable.

23. A respiratory therapy device according to claim 14, characterised in that the device includes an adjustable flow regulator arranged to regulate flow of air into the air inlet.

24. A respiratory therapy device according to claim 14, characterised in that the indicator flag that is displaced linearly or rotatably.

25. A respiratory therapy device according to claim 14, characterised in that indicator flag is arranged to be displaced continuously.

26. A respiratory therapy device according to claim 14, characterised in that the indicator flag is arranged to be displaced only when the displaceable wall is close to its limit of displacement towards the end of a breath.
